(19) United States
(12) Patent Application Publication
(10) Pub. No.: US 2005/0049494 A1
(43) Pub. Date: Mar. 3, 2005

(54) Method and apparatus for presenting multiple enhanced images

(76) Inventors: Arthur Gritzky, Pollham (AT); Josef Steininger, Vocklamarkt (AT)

Correspondence Address: Dean D. Small;Armstrong Teasdale LLP, Suite 2600, One Metropolitan Square, St. Louis, MO, 63102, US

(21) Appl. No.: 10/652,747

(22) Filed: Aug. 29, 2003

(51) Int. Cl. A61B 8/00

(52) U.S. Cl. 600/437; 600/453

(57) ABSTRACT

Method and apparatus for presenting multiple enhanced images of different anatomic features is provided. An ultrasonic volume data set having multiple anatomic features is acquired. Multiple enhanced images are presented simultaneously based on the multiple anatomic features within the data set.

### BACKGROUND OF THE INVENTION

This invention relates generally to diagnostic ultrasound systems. In particular, the present invention relates to method and apparatus for processing and displaying multiple enhanced images based on an identified plane within a volume of data.

Conventional ultrasound scanners are capable of acquiring and displaying a volume of data. Unfortunately, it has been difficult to display and compare different types and views of anatomic data within the same volume, such as an image viewed from the C-plane, or transverse to the series of scan planes comprising the volume. There is the possibility that important diagnostic data may be overlooked or missed by not processing or reviewing a portion of data, and extra time may be required to select and review multiple images.

Additionally, processing the C-plane data to enhance specific features, such as bone or soft tissue, for example, requires time and expertise on the part of the user. The user must be experienced and know the correct image processing protocol to use. Reprocessing data can be time consuming, and may result in longer exam times and perhaps a lower patient throughput. Furthermore, doctors who may be more familiar with reviewing image data from other modalities, such as X-ray, may find reviewing the ultrasound data more valuable if X-ray-like images could be created from the ultrasound volume for comparison with other processed images.

Thus, a system and method are desired to process and display C-plane data from within a volume that addresses the problems noted above and others previously experienced.

### BRIEF DESCRIPTION OF THE INVENTION

In one embodiment, a method for presenting multiple enhanced images of different anatomic features comprises acquiring an ultrasonic volume data set having multiple anatomic features. Multiple enhanced images are presented simultaneously. The multiple enhanced images are based on the multiple anatomic features within the volume data set.

In one embodiment, a method for presenting multiple enhanced images comprises acquiring a data set comprising volumetric data. Portions of the data set are processed with image enhancing techniques. Multiple images are presented based on the portions. Each of the multiple images is processed with a different image enhancing technique. The multiple images are presented simultaneously.

In one embodiment, a system for acquiring and presenting multiple enhanced images comprises a transducer for transmitting and receiving ultrasound signals to and from an area of interest. A receiver receives the ultrasound signals comprising a series of adjacent scan planes. The series of adjacent scan planes comprise a volumetric data set. A processor processes the series of adjacent scan planes and identifies portions of the volumetric data set being transverse to the series of adjacent scan planes. The processor processes the portions with image enhancing techniques. An output presents multiple images simultaneously. Each of the multiple images are processed with a different image enhancing technique.

### BRIEF DESCRIPTION OF THE DRAWINGS

FIG. 1 illustrates a block diagram of an ultrasound system formed in accordance with an embodiment of the present invention.

FIG. 2 illustrates an ultrasound system formed in accordance with an embodiment of the present invention.

FIG. 3 illustrates a real-time 4D volume acquired by the system of FIG. 2 in accordance with an embodiment of the present invention.

FIG. 4 illustrates a B-mode image and an enhanced image on the display in accordance with an embodiment of the present invention.

FIG. 5 illustrates a B-mode image with a plane of interest identified in accordance with an embodiment of the present invention.

FIG. 6 illustrates four enhanced images displayed simultaneously on the display in accordance with an embodiment of the present invention.

FIG. 7 illustrates multiple enhanced images based on the C-plane identified by the plane of FIG. 5 in accordance with an embodiment of the present invention.

FIG. 8 illustrates a block diagram of a portion of the ultrasound system of FIG. 2 in accordance with an embodiment of the present invention.

### DETAILED DESCRIPTION OF THE INVENTION

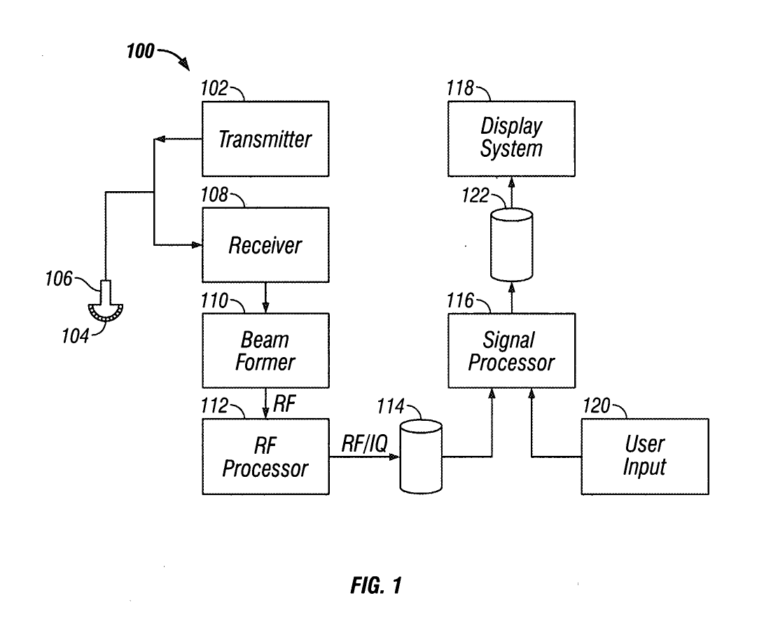

FIG. 1 illustrates a block diagram of an ultrasound system 100 formed in accordance with an embodiment of the present invention. The ultrasound system 100 includes a transmitter 102 which drives transducers 104 within a probe 106 to emit pulsed ultrasonic signals into a body. A variety of geometries may be used. The ultrasonic signals are back-scattered from structures in the body, like blood cells or muscular tissue, to produce echoes which return to the transducers 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to RF/IQ buffer 114 for temporary storage. A user input 120 may be used to input patient data, scan parameters, a change of scan mode, and the like.

The ultrasound system 100 also includes a signal processor 116 to process the acquired ultrasound information (i.e., RF signal data or IQ data pairs) and prepare frames of ultrasound information for display on display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation.

The ultrasound system 100 may continuously acquire ultrasound information at a frame rate that exceeds 50 frames per second—the approximate perception rate of the human eye. The acquired ultrasound information is displayed on the display system 118 at a slower frame-rate. An image buffer 122 is included for storing processed frames of acquired ultrasound information that are not scheduled to be displayed immediately. Preferably, the image buffer 122 is of sufficient capacity to store at least several seconds worth of frames of ultrasound information. The frames of ultrasound information are stored in a manner to facilitate retrieval thereof according to its order or time of acquisition. The image buffer 122 may comprise any known data storage medium.

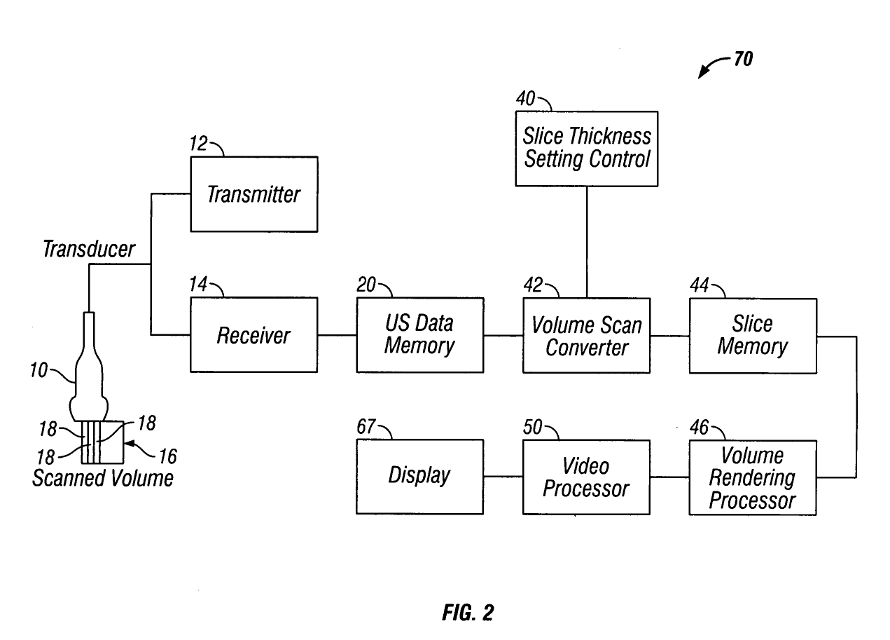

FIG. 2 illustrates an ultrasound system 70 formed in accordance with one embodiment of the present invention. The system 70 includes a probe 10 connected to a transmitter 12 and a receiver 14. The probe 10 transmits ultrasonic pulses and receives echoes from structures inside of a scanned ultrasound volume 16. Memory 20 stores ultrasound data from the receiver 14 derived from the scanned ultrasound volume 16. The volume 16 may be obtained by various techniques (e.g., 3D scanning, real-time 3D imaging, volume scanning, 2D scanning with transducers having positioning sensors, freehand scanning using a Voxel correlation technique, 2D or matrix array transducers and the like).

The transducer 10 is moved, such as along a linear or arcuate path, while scanning a region of interest (ROI). At each linear or arcuate position, the transducer 10 obtains scan planes 18. The scan planes 18 are collected for a thickness, such as from a group or set of adjacent scan planes 18. The scan planes 18 are stored in the memory 20, and then passed to a volume scan converter 42. In some embodiments, the transducer 10 may obtain lines instead of the scan planes 18, and the memory 20 may store lines obtained by the transducer 10 rather than the scan planes 18. The volume scan converter 20 may store lines obtained by the transducer 10 rather than the scan planes 18. The volume scan converter 42 receives a slice thickness setting from a control input 40, which identifies the thickness of a slice to be created from the scan planes 18. The volume scan converter 42 creates a data slice from multiple adjacent scan planes 18. The number of adjacent scan planes 18 that are obtained to form each data slice is dependent upon the thickness selected by slice thickness control input 40. The data slice is stored in slice memory 44 and is accessed by a volume rendering processor 46. The volume rendering processor 46 performs volume rendering upon the data slice. The output of the volume rendering processor 46 is passed to the video processor 50 and display 67.

The position of each echo signal sample (Voxel) is defined in terms of geometrical accuracy (i.e., the distance from one Voxel to the next) and ultrasonic response (and derived values from the ultrasonic response). Suitable ultrasonic responses include gray scale values, color flow values, and angio or power Doppler information.

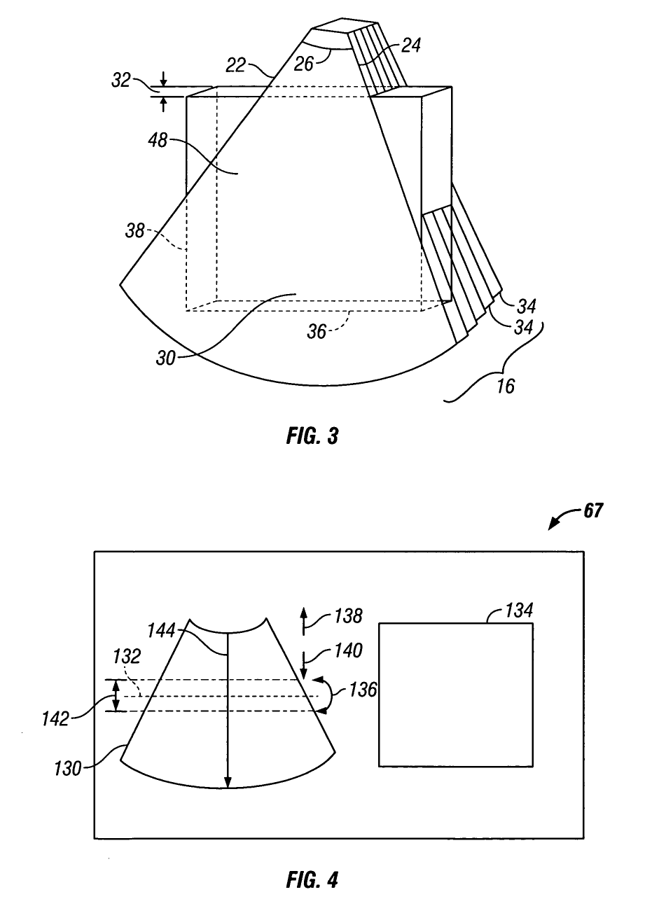

FIG. 3 illustrates a real-time 4D volume 16 acquired by the system 70 of FIG. 2 in accordance with one embodiment. The volume 16 includes a sector shaped cross-section with radial borders 22 and 24 diverging from one another at angle 26. The probe 10 electronically focuses and directs ultrasound firings longitudinally to scan along adjacent scan lines in each scan plane 18 and electronically or mechanically focuses and directs ultrasound firings laterally to scan adjacent scan planes 18. Scan planes 18 obtained by the probe 10, as illustrated in FIG. 2, are stored in memory 20 and are scan converted from spherical to Cartesian coordinates by the volume scan converter 42. A volume comprising multiple scan planes is output from the volume scan converter 42 and stored in the slice memory 44 as rendering box 30. The rendering box 30 in the slice memory 44 is formed from multiple adjacent image planes 34.

The rendering box 30 may be defined in size by an operator to have a slice thickness 32, width 36 and height 38. The volume scan converter 42 may be controlled by the slice thickness control input 40 to adjust the thickness parameter of the slice to form a rendering box 30 of the desired thickness. The rendering box 30 designates the portion of the scanned volume 16 that is volume rendered. The volume rendered processor 46 accesses the slice memory 44 and renders along the thickness 32 of the rendering box 30.

During operation, a 3D slice having a pre-defined, substantially constant thickness (also referred to as the rendering box 30) is acquired by the slice thickness setting control 40 (FIG. 2) and is processed in the volume scan converter 42 (FIG. 2). The echo data representing the rendering box 30 may be stored in slice memory 44. Predefined thicknesses between 2 mm and 20 mm are typical, however, thicknesses less than 2 mm or greater than 20 mm may also be suitable depending on the application and the size of the area to be scanned. The slice thickness setting control 40 may include a rotatable knob with discrete or continuous thickness settings.

The volume rendering processor 46 projects the rendering box 30 onto an image portion 48 of an image plane 34 (FIG. 3). Following processing in the volume rendering processor 46, the pixel data in the image portion 48 may pass through a video processor 50 and then to a display 67. The rendering box 30 may be located at any position and oriented at any direction within the scanned volume 16. In some situations, depending on the size of the region being scanned, it may be advantageous for the rendering box 30 to be only a small portion of the scanned volume 16.

FIG. 4 illustrates a B-mode image 130 having a depth 44 to one side of the display 67. Although the image being displayed is a B-mode image, a volume data set, such as volume 16 of adjacent image planes 34 (FIG. 3), has been acquired in real-time as discussed previously. A user may use the user input 120 to define a plane 132 of interest on the B-mode image 130. The plane 132 identifies a plane, such as the C-plane (i.e. anterior-to-posterior) through the volume data set having a minimum thickness of 0.1 mm. Therefore, the plane 132 defines a portion, or subset, of the data set or volume 16. The plane 132 may be radial, perpendicular, or at an intermediate angle with respect to the probe 10. Once the plane 132 has been identified, the user may rotate the plane 132 through an angle 136 with the user input 120. The user may also move the plane 132 up 138 towards the probe 10 or down 140 away from the probe 10.

The user may then select an image enhancement technique and/or other processing to be done to the volume data set identified by the plane 132. The image enhancement technique may be a volume rendering technique, for example. The user may wish to display image data associated with bone, and therefore selects an image enhancement technique based on this anatomic feature. Other anatomic features such as soft tissue and vessels may also be processed. For example, the user may use the user input 120 to select a volume rendering technique such as maximum density to display an enhanced image of bone. Alternatively, a subset of image enhancement techniques may be offered or suggested to the user based on the type of scan being performed, such as fetal scan, liver, and the like. The data set identified by the plane 132 is processed to create an enhanced image 134. The enhanced image 134 may be displayed in real-time on display 67 alone, such as in a larger format than illustrated in FIG. 4. Alternatively, the enhanced image 134 may be displayed on the display 67 simultaneously and in real-time with the B-mode image 130.

Additionally, the user may modify a thickness 142 of the volume data set. For example, the thickness 142 may be equidistant above and below the plane 132, or the plane 132 may identify the top or bottom of the thickness 142. The thickness 142 may or may not be displayed on display 67 as lines or in numerical format (not shown). In other words, varying the thickness 142 allows the user to view image data from multiple layers of the volume 30 that are parallel to the C-plane, or other plane 132, that the user has defined. The thickness 142 defined may be based on the image enhancement technique, the anatomic feature, the depth 144, and/or the acquisition type. If the user changes the position of the plane 132 after modifying the thickness 142, the size of the thickness 142 may be maintained. For example, if the user wishes to display an enhanced image 134 based on bone, a thicker thickness 142 is defined. If the user wishes to display an enhanced image 134 based on vessels, a thinner thickness 142 is defined.

Changes made by the user to the position of the plane 132 and the thickness 142 may be displayed in real-time. Therefore, the enhanced image 134 is updated as the plane 132 and/or thickness 142 are varied. Therefore, a user may continue to modify the thickness 142 and move the plane 132 until the desired enhanced image 134 is displayed.

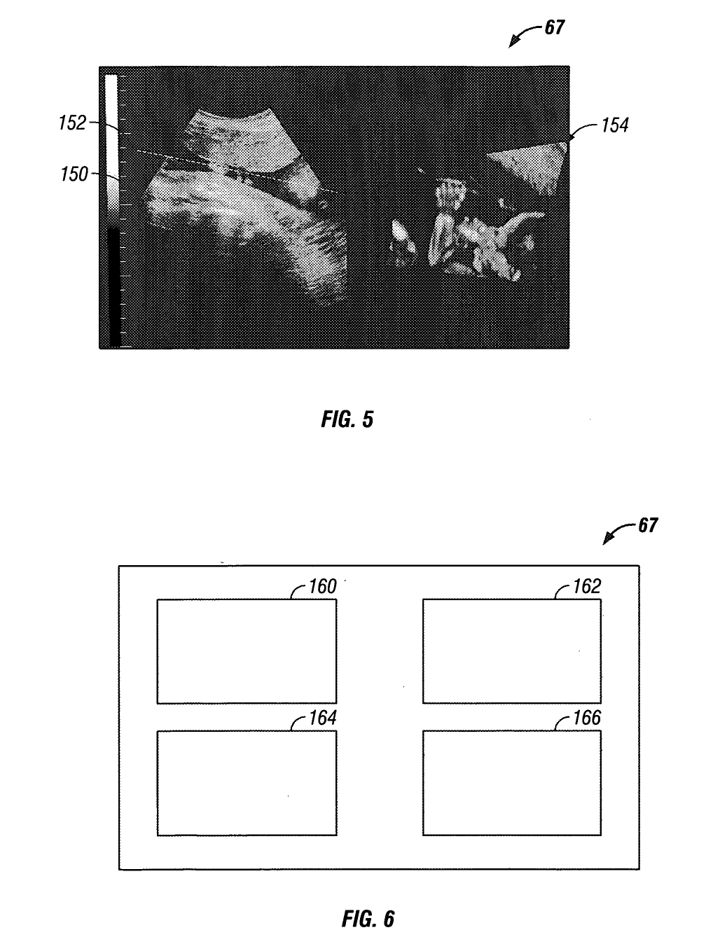

FIG. 5 illustrates a B-mode image 150 with plane 152 identifying a plane of interest. The plane 152 may define the C-plane as previously discussed. The B-mode image 150 provides a frame of reference for the user, allowing the user to identify the plane 152 based on real-time data. By way of example only, the B-mode image 150 in FIG. 5 illustrates a fetus. It should be understood that other anatomy may be scanned and processed, such as the liver, heart, kidneys, and the like.

An enhanced image 154 corresponding to the plane 152 is illustrated simultaneously on the display 67 with the B-mode image 150. In this example, the user has selected the plane 152 to display a C-plane image of the fetal arms using a volume contrast imaging technique, such as maximum density. The size of the thickness 142 may be increased or decreased as discussed previously.

FIG. 6 illustrates four enhanced images 160-166 displayed simultaneously on the display 67. Each of the enhanced images 160-166 have been processed according to a predefined set of image enhancing techniques, and correspond to a plane of data, such as the plane 132 of FIG. 4.

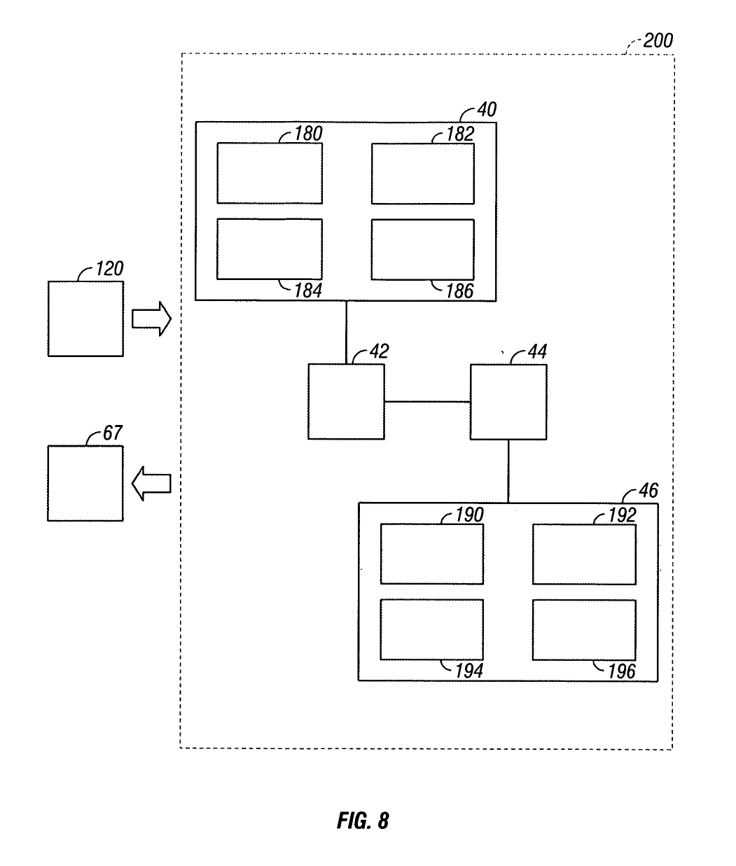

FIG. 8 illustrates a block diagram of a portion 200 of the ultrasound system 70 of FIG. 2. In FIG. 8, the slice thickness setting control 40 includes four individual thickness controls 180-186. The volume rendering processor 46 includes four individual rendering setting controls 190-196. It should be understood that FIG. 8 is a conceptual representation only. For example, a single slice thickness setting control 40 may be used to set multiple different slice thicknesses 142 simultaneously, and a single volume rendering processor 46 may be used to set the different rendering techniques and process multiple volumes of data simultaneously.

When the user begins to acquire the B-mode volume data set, the type of scan being performed, such as of a fetus, a liver, and the like, is identified through the user input 120. The user also adjusts the depth 144 of the scan to include the desired information within the B-mode image. The operator then defines the plane 132, as discussed previously with FIG. 4. Although the following discussion is limited to acquiring 3-D and 4-D B-mode volumetric data, it should be understood that other acquisition modes may be used, such as conventional grayscale sonography, B-flow, harmonic and co-harmonic sonography, color Doppler, tissue harmonic imaging, pulse inversion harmonic imaging, Power Doppler, and tissue Doppler.

Depending upon the acquisition type, a different subset of anatomic features, associated with a different subset of image enhancing techniques, may be expected. For example, when scanning a fetus, the subset of anatomic features may include bone, vessel, contrast, and soft tissue, which have known ultrasound characteristic responses. When scanning a liver, however, the system 70 may not include bone in the subset of anatomic features. In addition, the depth 144 of the scan also impacts the thickness 142 which is associated with the image enhancing techniques.

The user may then initiate the automatic processing of the four enhanced images 160-166 through the user input 120. For example, the user input 120 may comprise a single protocol or button selection. A subset of anatomic features having associated image enhancing techniques has been predefined. The subset may provide a default, which is applied when scanning any anatomy. Alternatively, the subset of anatomic features may be based on one or more of the type of acquisition, probe type, the depth 144, and the like. The thickness controls 180-186 of the slice thickness setting control 40 automatically set the predefined subset of anatomic features. Therefore, each of the thicknesses 142 for the different enhanced images 160-166 include at least a common subset of the data set. The rendering setting controls 190-196 of the volume rendering processor 46 automatically identify the appropriate image enhancing techniques, and the volume rendering processor 46 processes the slice data identified by the respective thickness controls 180-186. The enhanced images 160-166 are then displayed on display 67. Therefore, the correct thickness 142 of each enhanced image 160-166 is automatically defined for the user, so there is no need for the user to manually vary the thickness 142 to display enhanced images of different anatomic features.

For example, enhanced image 160 may use a “bone” anatomic feature setting. With this setting, the thickness control 180 automatically defines the thickness 142, such as between 10-15 mm. The rendering setting control 190 identifies the correct technique, such as a maximum density rendering technique, and the volume rendering processor 46 processes the layers of the volume 30 that are parallel to the plane 132 and within the thickness 142. Enhanced image 162 may use a “soft tissue” anatomic feature setting. With this setting, the thickness control 182 identifies the thickness 142, which may be approximately 3 mm. The rendering setting control 192 identifies the correct technique, such as an X-ray rendering technique, and the volume rendering processor 46 processes the layers of the volume 30 that are parallel to the plane 132 and within the thickness 142. The X-ray rendering technique may be used to provide an image comparable to an slice image created when using X-ray radiation. This technique may also be called average projection. Other rendering modes may be used to enhance anatomic features, such as gradient light rendering and maximum transparency. Additionally, other image processing techniques may be used to process and create enhanced images.

Similarly, enhanced images 164 and 166 may use “contrast” and “vessels” anatomic feature settings, respectively. The thickness controls 184 and 186 identify the thicknesses 142 (by way of example only, 1 mm with threshold low 0, and 5-10 mm, respectively) and the rendering setting controls 194 and 196 identify the techniques (by way of example only, surface and minimum density rendering techniques, respectively). The volume rendering processor 46 processes the layers of the volume 30 that are parallel to the plane 132 and within the thicknesses 142 for each of the enhanced images 164 and 166.

The enhanced images 160-166 are displayed simultaneously on display 67. It should be understood that although the aforementioned process was discussed as creating the enhanced images 160-166 singularly, the enhanced images 160-166 may be created at the same time. Therefore, multiple anatomic features may be enhanced and displayed on the display 67, and be contrasted with respect to each other at the same time.

Therefore, the displaying and processing of the volume data set is automatically performed by predefining the subset of anatomic features within the volume data set to be processed, and identifying the associated subset of image enhancing techniques. The user does not have to choose the correct image enhancing technique nor define the correct thickness 142 for the scan to display the desired enhanced image 160-166 of an anatomic feature. Additionally, by automatically displaying multiple enhanced images 160-166 based on the same C-plane volume data set, where the enhanced images 160-166 include at least a common subset of the data set, the images comprising different anatomic features of the same plane 132 (C-plane) may be easily compared. Thus, by presenting the processed information automatically, it is less likely that valuable diagnostic data may not be displayed or may be overlooked. Also, the user input, such as the number of key strokes and other required entries, is greatly simplified, and the time required to manually process the enhanced images 160-166 is eliminated.

Alternatively, the user may predefine the different anatomic features they wish to have automatically identified and processed. The user's predefined subset of anatomic features and the associated image enhancing techniques may be based on the acquisition type, probe type, and/or personal preference and the like. It should be understood that although four enhanced images 160-166 are illustrated in FIG. 6, more or less enhanced images 160-166 may be displayed based on the size of the display 67, user preference, and the like.

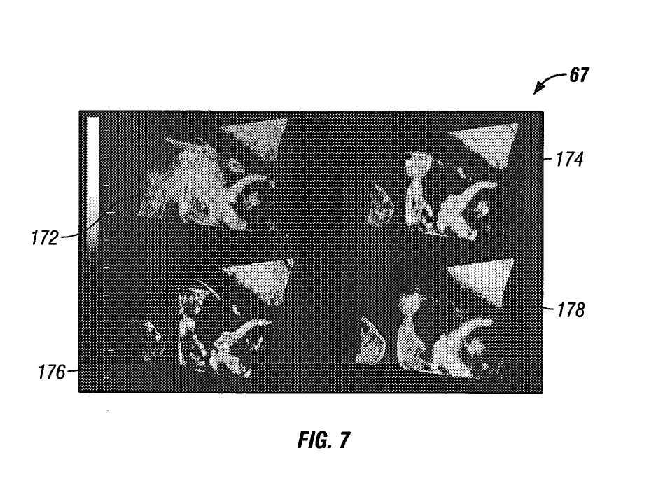

FIG. 7 illustrates multiple enhanced images 172-178 based on a C-plane, such as the C-plane identified by plane 152 of FIG. 5. After the user identifies the type of scan and plane 152, enhanced images 172-178 are automatically processed and displayed. Enhanced image 172 is processed using the bone anatomic feature setting, or the maximum density rendering technique. Enhanced image 174 is processed using the soft tissue anatomic feature setting, or the X-ray rendering technique. Enhanced image 176 is processed using the contrast anatomic feature setting, or the surface rendering technique. Enhanced image 178 is processed using the vessels anatomic feature setting, and the minimum density rendering technique. The enhanced images 172-178 are displayed simultaneously on display 67.

The enhanced images 172-178 may be displayed in real-time as the volume 30 is being acquired. In this embodiment, the B-mode image 150 may be displayed on a different display 67, not displayed, or displayed in place of or in addition to, one of the enhanced images 172-178. Alternatively, the volume 30 may be acquired and stored prior to creating the enhanced images 172-178. It should be understood that although FIGS. 5 and 7 utilize volume rendering techniques as the image enhancing technique, other image enhancing techniques may be used to process enhanced images 154 and 172-178.

While the invention has been described in terms of various specific embodiments, those skilled in the art will recognize that the invention can be practiced with modification within the spirit and scope of the claims.

1. A method for presenting multiple enhanced images of different anatomic features, comprising:
acquiring an ultrasonic volume data set having multiple anatomic features; and
presenting multiple enhanced images simultaneously, said multiple enhanced images being based on said multiple anatomic features within said volume data set.

2. The method of claim 1, said anatomic features comprising at least one of bone, soft tissue, contrast and vessels.

3. The method of claim 1, further comprising selecting volume rendering techniques, said multiple enhanced images being based on said volume rendering techniques.

4. The method of claim 1, further comprising:
identifying a plane within said data set; and
identifying at least one thickness of said plane, said multiple enhanced images being based on said at least one thickness of said plane.

5. The method of claim 1, further comprising processing said volume data set with predefined image enhancing techniques, each of said multiple enhanced images being processed with a different image enhancing technique.

6. The method of claim 1, further comprising:
said processing step further comprising processing said volume data set in real-time while receiving real-time ultrasonic information; and
said presenting step further comprising presenting said multiple enhanced images in real-time.

7. The method of claim 1, further comprising selecting volume rendering techniques to enhance said multiple anatomic features, said volume rendering techniques being one of surface texture, maximum density, minimum density, average projection, gradient light rendering, and maximum transparency

8. The method of claim 1, further comprising:
identifying a plane within said volume data set;
identifying thicknesses of said plane for each of said multiple enhanced images; and
processing said data set based on said thicknesses, each of said multiple enhanced images being based on a different thickness.

9. A method for presenting multiple enhanced images, comprising:
acquiring a data set comprising volumetric data;
processing portions of said data set with image enhancing techniques; and
presenting multiple images based on said portions, each of said multiple images being processed with a different image enhancing technique, said multiple images being presented simultaneously.

10. The method of claim 9, said acquiring step further comprising acquiring said data set using at least one of the following acquisition modes: 3-D volume, 4-D volume, conventional grayscale sonography, B-flow, color Doppler, tissue Doppler, Power Doppler, and harmonic and co-harmonic sonography.

11. The method of claim 9, further comprising identifying a plane, said plane being a C-plane with respect to said volumetric data, said portions of said data set being based on said plane.

12. The method of claim 9, further comprising:
identifying a plane within said data set; and
identifying a depth based on said data set, said portions being based on said plane and having different thicknesses based on at least one of said depth and said different image enhancing techniques.

13. The method of claim 9, said data set further comprising anatomic features, said anatomic features being one of bone, soft tissue, contrast, and vessel, said image enhancing techniques being used to enhance said anatomic features.

14. The method of claim 9, said image enhancing techniques being one of surface texture, maximum density, minimum density, and average projection.

15. The method of claim 9, further comprising:
identifying an acquisition type; and
predefining a subset of said image enhancing techniques based on said acquisition type.

16. The method of claim 9, said data set further comprising at least one of ultrasonic data, MR data, and CT data.

17. A system for acquiring and presenting multiple enhanced images, comprising:
a transducer for transmitting and receiving ultrasound signals to and from an area of interest;
a receiver for receiving said ultrasound signals comprising a series of adjacent scan planes comprising a volumetric data set;
a processor for processing said series of adjacent scan planes, said processor identifying portions of said volumetric data set being transverse to said series of adjacent scan planes, said processor processing said portions with image enhancing techniques; and
an output for presenting multiple images simultaneously, each of said multiple images being processed with a different image enhancing technique.

18. The system of claim 17, wherein each of said portions comprises at least a common subset of said volumetric data set.

19. The system of claim 17, further comprising:
an input for identifying a plane within said volumetric data set;
said processor identifying a depth based on said volumetric data set; and
at least one thickness control setting a thickness of each of said portions being based on at least one of said depth and said image enhancing techniques.

20. The system of claim 17, further comprising:
an input for receiving an acquisition type; and
said processor further comprising identifying a subset of said image enhancing techniques based on said acquisition type.

21. The system of claim 17, further comprising an input for predefining at least one subset of said image enhancing techniques, said processor using said at least one subset to process said multiple images.

22. The system of claim 17, further comprising:
an input for receiving an acquisition type; and
said transducer further comprising having a transducer type, said processor further comprising identifying a subset of said image enhancing techniques based on said transducer type.

23. The system of claim 17, further comprising:
a memory for storing said volumetric data set; and
said processor further comprising retrieving said volumetric data set from said memory prior to said processing.

24. The system of claim 17, further comprising at least one rendering setting control for identifying said image enhancing techniques.
